(19) United States
(12) Patent Application Publication
(10) Pub. No.: US 2025/0128033 A1
(43) Pub. Date: Apr. 24, 2025

(54) CEREBROSPINAL FLUID ACCESS DEVICES AND METHODS

(71) Applicants: Kuo Chao, Los Angeles, CA (US); Wei Chao, Cupertino, CA (US)

(72) Inventors: Kuo Chao, Los Angeles, CA (US); Wei Chao, Cupertino, CA (US)

(21) Appl. No.: 18/922,344

(22) Filed: Oct. 21, 2024

Related U.S. Application Data
(60) Provisional application No. 63/580,408, filed on Oct. 23, 2023.

(51) Int. Cl. A61M 27/00

(52) U.S. Cl. CPC A61M 27/006

(57) ABSTRACT

A device for accessing a cerebrospinal fluid of a patient is provided. The device can include a central conduit that defines a central bore, which extends from a proximal end of the device to a distal end of the device. The device can further include a shunting port on the central conduit and within a longitudinal distance of 6 mm from the distal end of the device. The device further include a sealing membrane at the proximal end that forms a fluidic seal at the proximal end of the central bore.

### CROSS-REFERENCE TO RELATED APPLICATION

This application claims priority to U.S. Provisional Application No. 63/580,408, filed Oct. 23, 2023, and entitled “CSF INTERVENTIONS,” the disclosure of which application is hereby incorporated by reference in its entirety.

### BACKGROUND

Cerebrospinal fluid (CSF) is distributed around, and provides cushioning to, the brain and spinal cord. Access to a patient's CSF can be useful for diagnosing and treating a variety of physiological maladies. For example, infections such as spinal meningitis can be detected by sampling the CSF of a patient. The CSF space is also a convenient access point for introducing therapies that need to cross the blood-brain barrier, such as, for example chemotherapy agents.

Current methods of accessing the CSF are decades old and suffer from numerous deficiencies. The devices and methods disclosed herein are directed to novel ways of accessing the CSF of a person. The benefits of the devices and methods disclosed herein include increased patient comfort and increased patient safety compared to existing methods of accessing the CSF.

### SUMMARY

Disclosed herein are embodiments of a CSF-access device. In certain arrangements, the CSF-access device can include a central bore that extends from a proximal end of the device to a distal end of the device. A lateral channel in the wall of the device can connect fluidically a shunting port disposed at the exterior surface of the wall of the device to the central bore. A delivery assembly disposed within the central bore can have an axial bore and a lateral channel disposed radially outward from the axial bore, the lateral channel fluidically isolated from the axial bore. In some arrangements, the device can further include an epidural-blocking structure disposed on the exterior surface of the wall and extending radially away from central bore. In some arrangements, the device can further include a tissue anchor distal of the epidural-blocking structure, the tissue anchor being configured, in some arrangements, to deploy from a low-profile configuration to a high-profile configuration upon the delivery assembly being removed from the device.

Also disclosed are methods of implanting a CSF-access device in a patient. In some arrangements, the CSF-access device can be implanted using an over-the-wire delivery assembly that passes through a central bore of the device. In some arrangements, the delivery assembly can comprise a coaxial arrangement of a guidewire within a delivery needle. In some arrangements, the delivery assembly can include a piercing stylet disposed within the delivery needle and configured to pierce a dura of the patient to gain access to a CSF space of the patient.

Also disclosed are methods of removing a CSF-access device from a patient. In some arrangements, the proximal end of the device can include a magnetic collar configured to guide and otherwise facilitate coupling of the proximal end of the device to a distal end of a device-retrieval assembly. In some arrangements, the proximal end of the CSF-access device can include a thread or groove that couples to a corresponding groove or thread of a device-retrieval assembly to allow the device-retrieval assembly to secure a coupling between the device and the device-retrieval assembly for removal of the device from the patient.

Also disclosed are methods of closing an access site after removing a CSF-access device from the access site. In some arrangements, a device-closure assembly can include a balloon port configured to inflate a balloon and a sealant port configured to deliver a sealant to a distal end of the closure device. In some aspects, the device-closure assembly can be configured to inflate the balloon and seal the dura to prevent a sealant introduced through the sealant port from gaining access to the CSF-space. Upon the sealant curing, the balloon can be deflated and removed from the CSF-space in a low-profile configuration.

### BRIEF DESCRIPTION OF THE DRAWINGS

Embodiments of the present disclosure will now be described hereinafter, by way of example only, with reference to the accompanying drawings in which:

FIG. 1 illustrates a prior art method of accessing the CSF of a patient.

FIG. 2 illustrates a prior art device for implanting a CSF drain.

FIG. 3 illustrates a CSF-access device implanted in a patient, according to some aspects of the present disclosure.

FIG. 4 illustrates a dilator configured to dilate a CSF-access device, according to some aspects of the present disclosure.

FIG. 5A illustrates a CSF-access device mounted on a delivery assembly, according to some aspects of the present disclosure.

FIG. 5B illustrates a CSF-access device implanted in a patient to establish an access to a CSF space of the patient, according to some aspects of the present disclosure.

FIG. 5C illustrates a CSF-access device implanted in a patient to establish an access to an intravenous space of the patient, according to some aspects of the present disclosure.

FIG. 6A illustrates a proximal hub of a CSF-access device sutured to an outer surface of a patient's skin, according to some aspects of the present disclosure.

FIG. 6B illustrates a proximal hub of a CSF-access device sutured subcutaneously to a patient's skin, according to some aspects of the present disclosure.

FIG. 6C illustrates a CSF-access device, according to some aspects of the present disclosure.

FIG. 6D shows a top view of the proximal end of the CSF-access device of FIG. 6C, according to some aspects of the present disclosure.

FIG. 6E illustrates a side cross-sectional view of a proximal end of a CSF-access device, according to some aspects of the present disclosure.

FIG. 6F illustrates a side cross-sectional view of a proximal end of a CSF-access device, according to some aspects of the present disclosure.

FIG. 7A illustrates a CSF-access device with a proximal hub of the device secured to an outer surface of a patient's skin, according to some aspects of the present disclosure.

FIG. 7B illustrates a CSF-access device with a proximal hub of the device secured to an outer surface of a patient's skin, according to some aspects of the present disclosure.

FIG. 7C illustrates a CSF-access device mounted on a delivery assembly, according to some aspects of the present disclosure.

FIG. 7D illustrates a cross-sectional view of a CSF-access device mounted on a delivery assembly, according to some aspects of the present disclosure.

FIG. 7E illustrates a cross-sectional view of a CSF-access device implanted in a patient to establish an access to a CSF space of the patient, according to some aspects of the present disclosure.

FIG. 8A illustrates a cross-sectional view of a CSF-access device mounted on a delivery assembly, according to some aspects of the present disclosure.

FIG. 8B illustrates a cross-sectional view of a CSF-access device mounted on a delivery assembly, according to some aspects of the present disclosure.

FIG. 8C illustrates a cross-sectional view of a CSF-access device implanted in a patient to establish an access to a CSF space of the patient, according to some aspects of the present disclosure.

FIG. 9A illustrates a CSF-access device implanted in a patient to establish an access to a CSF space of the patient, according to some aspects of the present disclosure.

FIG. 9B illustrates a CSF-access device, according to some aspects of the present disclosure.

FIG. 9C illustrates a CSF-access device, according to some aspects of the present disclosure.

FIG. 9D illustrates a CSF-access device mounted on a delivery assembly, according to some aspects of the present disclosure.

FIG. 9E illustrates a cross-sectional view of a delivery assembly for implanting a CSF-access device in a patient to establish an access to a CSF space of the patient, according to some aspects of the present disclosure.

FIG. 9F illustrates an axial view of the delivery assembly of FIG. 9E, according to some aspects of the present disclosure.

FIG. 9G illustrates a cross-sectional view of a delivery assembly configured for implanting a CSF-access device, according to some aspects of the present disclosure.

FIG. 9H illustrates a cross-sectional view of a proximal end of a delivery assembly configured for implanting a CSF-access device, according to some aspects of the present disclosure.

FIG. 10A illustrates a cross-sectional view of a CSF-access device mounted on a delivery assembly, according to some aspects of the present disclosure.

FIG. 10B illustrates a cross-sectional view of a CSF-access device implanted in a patient to gain access to a CSF space of the patient, according to some aspects of the present disclosure.

FIG. 10C illustrates a cross-sectional view of a CSF-access device mounted on a delivery assembly and positioned within the patient to gain access to the CSF space of the patient, according to some aspects of the present disclosure.

FIG. 10D illustrates a cross-sectional view of a CSF-access device implanted in a patient, according to some aspects of the present disclosure.

FIG. 11 illustrates a cross-sectional view of a CSF-access device implanted in a patient to establish access to a CSF space of the patient, according to some aspects of the present disclosure.

FIG. 12A illustrates a cross-sectional view of a CSF-access device implanted in a patient, according to some aspects of the present disclosure.

FIG. 12B illustrates a cross-sectional view of a retrieval assembly coupled to a CSF-access device implanted in a patient, according to some aspects of the present disclosure.

FIG. 12C illustrates a retrieval assembly coupled to a CSF-access device, according to some aspects of the present disclosure.

FIG. 12D illustrates a retrieval assembly coupled to a CSF-access device, according to some aspects of the present disclosure.

FIG. 13 illustrates a closure device that can be used to repair tissue damage of a patient following removal of a CSF-access device from the patient, according to some aspects of the present disclosure.

FIG. 14 illustrates a method of implanting a CSF-access device, according to some aspects of the present disclosure.

### DETAILED DESCRIPTION

### Overview

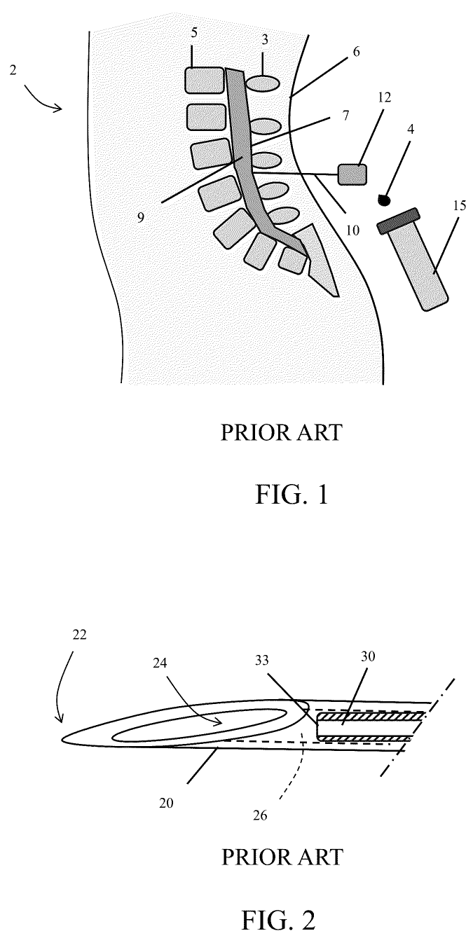

This disclosure relates generally to devices and methods for accessing a cerebrospinal fluid (CSF) of a patient. FIG. 1 illustrates a prior art method of accessing the CSF of a patient. A prior art method of accessing the CSF of a patient 2 can include having the patient 2 lay on the patient's side with the back of the patient 2 facing a medical practitioner. The medical practitioner can access the CSF of the patient 2 by inserting a needle 10 through the skin 6 of the patient 2, passing the needle 10 between two adjacent vertebrae 3, and penetrating the dura 7 to access the CSF space 9 of the patient 2. The proximal end 12 of the needle 10 (i.e., the end of the needle 10 that closest to the medical practitioner and most distant from the patient) can be configured to allow a CSF sample 4 to be collected from the CSF space 9 and into a sample-collection vessel 15.

FIG. 2 illustrates a prior art method for implanting a CSF drain 30 into a patient. The drain 30 can be housed within the bore 26 of a delivery needle 20. The delivery needle 20 can have a piercing tip 22 configured to pierce the dura 7 (FIG. 1) of a patient to gain access to the CSF space 9. Once the distal opening 24 of the needle 20 has crossed the dura 7 and entered the CSF space 9, the distal end 33 of the drain 30 can be advanced through the distal opening 24 of the delivery needle 20 and into the CSF space 9. After the distal end 33 of the drain 30 is placed within the CSF space 9, the delivery needle 20 can be withdrawn, leaving the distal end 33 of the drain 30 in the CSF space 9 of the patient, thereby establishing access to the CSF space 9 by establishing a fluid pathway between the CSF space 9 and the proximal end (not shown) of the drain 30.

One problem with the prior art method of implanting a CSF drain 30 is that the prior art method requires the bore 26 of the delivery needle 20 to have a large diameter to house the drain 30 during implantation of the drain 30 across the dura 7. As described herein, the devices and methods of the present disclosure achieve access to the CSF space 9 in a minimally-invasive manner compared to prior art methods. With reference to the prior art method depicted in FIG. 1, a problem with existing CSF drains is that the proximal end 12 of the CSF drain is exposed, which could place the patient at risk of infection. As described herein, the devices and methods of the present disclosure can allow, in some arrangements, a subcutaneous implantation of a proximal end of a CSF-access device. Subcutaneous implantation of the proximal end can increase patient safety by reducing the risk of infection through an exposed proximal end. In some configurations the proximal end of the present CSF-access devices can be configured to allow repeated access to the CSF space 9 without requiring repeated punctures of the dura 7, as described herein.

### CSF-Access Device

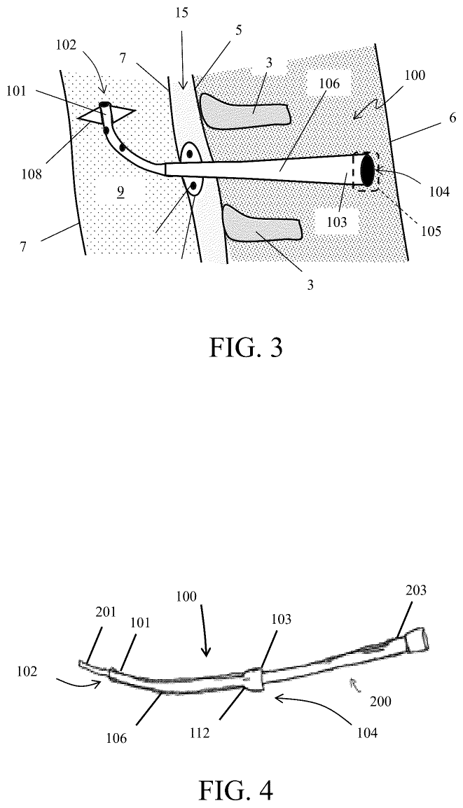

FIG. 3 depicts an illustrative, non-limiting example of a CSF-access device 100, according to some aspects of the present disclosure. FIG. 3 shows that in some aspects the CSF-access device 100 can be configured similar to a port through which one can gain access to a CSF space 9 of the patient. In some arrangements, the CSF-access device 100 can be configured similarly to a shunt, or to a drain, or to a pump, or to a sheath, as described herein. In some arrangements, the CSF-access device 100 can be configured for a minimally-invasive, over-the-wire implantation into the patient, as described herein. In some aspects, the CSF-access device 100 can include a distal end 101 configured to reside in the CSF space 9 of the patient following implantation of the CSF-access device 100. The CSF-access device 100 can include a proximal end 103, which in FIG. 3 is shown to terminate within a subcutaneous space of the patient. However, as discussed herein, the CSF-access device 100 can, in some aspects, have the proximal end 103 exposed external to the skin 6 of the patient following implantation of the CSF-access device 100.

The terms “distal” and “proximal” are used herein merely for the sake of simplicity in providing a spatial reference for different portions of the CSF-access device 100. The terms are meant to reflect spatial proximity relative to a medical practitioner (proximal) who is performing a medical procedure on a patient (distal). For example, a hypodermic syringe would be considered to have a needle at the distal end of the syringe, while the plunger end of the syringe would be considered to be the proximal end of the syringe because when the syringe is used by a medical practitioner to perform a medical procedure on a patient, the needle end (distal) is toward the patient, and the plunger end (proximal) is near the medical practitioner. In FIG. 3, the CSF-access device 100 has been implanted in a patient by advancing, as described herein, the distal end 101 through the skin 6 of the patient, passing the distal end 101 between two adjacent vertebrae 3, crossing the distal end 101 through the ligamentum flavum 5, advancing the distal end 101 through the epidural space 15 to reach the dura 7, and crossing the distal end 101 through the dura 7 to bring the distal end 101 into the CSF space 9 of the patient. In the illustrated port-configured variant of the CSF-access device 100, the proximal end 103 resides subcutaneously within the tissue of the patient between the skin 6 and the ligamentum flavum 5. In some aspects, the CSF-access device 100 can be configured to have the proximal end 103 of the implanted CSF-access device 100 reside outside of the skin 6, or to reside deeper within the skin 6 (e.g., between two adjacent vertebrae 3), as described herein.

With continued reference to FIG. 3, the CSF-access device 100 can include in some configurations a distal-end opening 102 and a proximal-end opening 104. In some aspects, a central conduit 106 can extend between the distal-end opening 102 and the proximal-end opening 104. The central conduit 106 can define a flow passage that communicates between the distal-end opening 102 and the proximal-end opening 104. In the illustrated device 100, the central conduit 106 has a form that can be characterized as a proximally-diverging-cylindrical tube. The central conduit 106 can have an axial cross-section defined by the shape of the bore of the central conduit 106 at a fixed axial position along the central conduit 106. For example, a central conduit 106 that is shaped like a cylindrical tube would have a circular axial cross-section at each fixed location along the entire length of the tube. In some aspects, the central conduit 106 can have a circular axial cross-section. In some arrangements, the central conduit 106 can have an axial cross-section that has a shape other than circular (e.g., ellipse, oval, rectangular, triangular, polyhedral). In some aspects, the conduit 106 can have an axial cross-section that changes in shape or size along the longitudinal length of the conduit 106. In some arrangements, the conduit 106 can diverge in the proximal direction, i.e., the axial cross-sections of the conduit 106 increase in size as one proceeds along the conduit 106 in a direction from the distal end 101 to the proximal end 103. In some arrangements, the proximal end 103 can be ellipsoidal to give the proximal end 103 a reduced-height profile for a given axial cross-sectional area, thereby increasing patient comfort when the proximal end 103 is embedded in the patient's skin 6.

As shown in FIG. 3, the CSF-access device 100 can include a tissue anchor 108 at the distal end 101 of the device 100, in some arrangements. However, as described herein, the CSF-access device 100 can be configured to include a distal end 101 that does not include a tissue anchor 108. In FIG. 3, the tissue anchor 108 is shown as a Malekot-type tissue anchor, but the tissue anchor 108 can be alternatively fashioned, such as, for example a pig-tail anchor. The proximal end 103 can include a sealing membrane 105 (shown as a dashed line in FIG. 3). The scaling membrane 105 can be configured to cover and seal the proximal-end opening 104. The sealing membrane 105 can reduce the risk of infection being introduced to the patient, for example, through the conduit 106 to the CSF space 9. In some aspects, the sealing membrane 105 can be configured to prevent CSF from flowing out of the proximal-end opening 104. In some configurations, the sealing membrane 105 can be a silicone material, or similar material, that can be penetrated by a sharp instrument (e.g., needle). In some aspects, the sealing membrane 105 can allow the proximal-end opening 104 to be implanted subcutaneously while maintaining a fluid-tight seal at the proximal-end opening 104. With reference to FIG. 3, the skilled artisan will appreciate that a subcutaneously-implanted proximal-end 104 having a sealing membrane 105 can allow repeated access to the CSF space 9 through the sealing membrane 105 (e.g., by repeated needle puncture across, and subsequent needle removal from, the sealing membrane 105) that avoids repeated injury (e.g., puncture) to the dura 7 while maintaining the fluidic integrity (e.g., a fluid-tight seal) of the CSF space 9.

FIG. 4 depicts an illustrative, non-limiting example of a delivery apparatus 200 for use with a CSF-access device 100, according to some aspects of the present disclosure. The delivery apparatus 200 can be configured similar to a dilator to assist with the implantation of a CSF-access device 100, as described herein. Similar to the CSF-access device 100, the delivery apparatus 200 can have a distal end 201 and a proximal end 203. FIG. 4 shows the CSF-access device 100 can be configured to receive the delivery apparatus 200 within the central conduit 106 of the device 100. In the illustrated example, the delivery apparatus 200 is shown after the distal end 201 of the delivery apparatus 200 has entered the central conduit 106 through the proximal-end opening 104 of the CSF-access device 100 and has been advanced distally along the central conduit 106 until the distal end 201 has emerged through the distal-end opening 102 such that the distal end 201 of the delivery apparatus 200 is distal to the distal end 101 of the CSF-access device 100.

As can be appreciated from FIG. 4, in some arrangements the delivery apparatus 200 can be configured as a dilator that is sized to distort (e.g., expand) the shape of the central conduit 106 as the delivery apparatus 200 is advanced toward and through the distal-end opening 102. For example, the diameter of the delivery apparatus 200 at its distal end 201 can increase in the proximal direction (i.e., toward the proximal end 203). This proximally-increasing diameter of the delivery apparatus 200 can force the central conduit 106 to stretch as the delivery apparatus 200 is advanced distally through the distal opening 102. In some aspects, the CSF-access device 100 can minimize damage to the dura 7 by allowing the medical practitioner to implant the CSF-access device 100 through a small puncture of the dura 7 that is subsequently dilated by the delivery apparatus 200 as opposed to the prior art method that requires a large puncture of the dura 7. In some aspects, the delivery apparatus 200 can modify the CSF-access device 100 to allow better access to the CSF space 9. For example, the delivery apparatus 200 can be sized to expand the axial cross-sectional area of the central conduit 106 in region of the central conduit 106 that is proximal and adjacent to the region of the central conduit 106 that spans the dura 7. For a given puncture hole in the dura 7 and a given fluid pressure in the CSF space 9, the aforementioned proximally-divergent central conduit 106 will allow more flow of CSF fluid compared to a central conduit 106 that does not diverge proximally after crossing the dura 7. In this way, the CSF-access device 100 can allow improved results (i.e., access to the CSF space 9) with reduced tissue damage (e.g., size of puncture hole formed in the dura 7).

In some configurations, the delivery apparatus 200 can be sized to form a proximal collar 112 in the central conduit 106 near the proximal end 103 as the delivery apparatus 200 is advanced distally through the central conduit 106. For example, a stent-like ring (not shown) could be embedded in the wall of the central conduit 106 in an initial low-profile configuration with a first diameter. Advancing distally through the ring a delivery apparatus 200 with a proximally-diverging axial cross-sectional diameter can force the stent-like ring to deform into a deployed large-diameter configuration. In some arrangements, the distal collar 112 can be formed in the central conduit 106 without requiring a delivery apparatus 200 to distort the central conduit 106, such as, for example, by molding a distal collar 112 (e.g., protrusion) onto the outer surface of the central conduit 106 during manufacture of the CSF-access device 100.

### Port Configuration

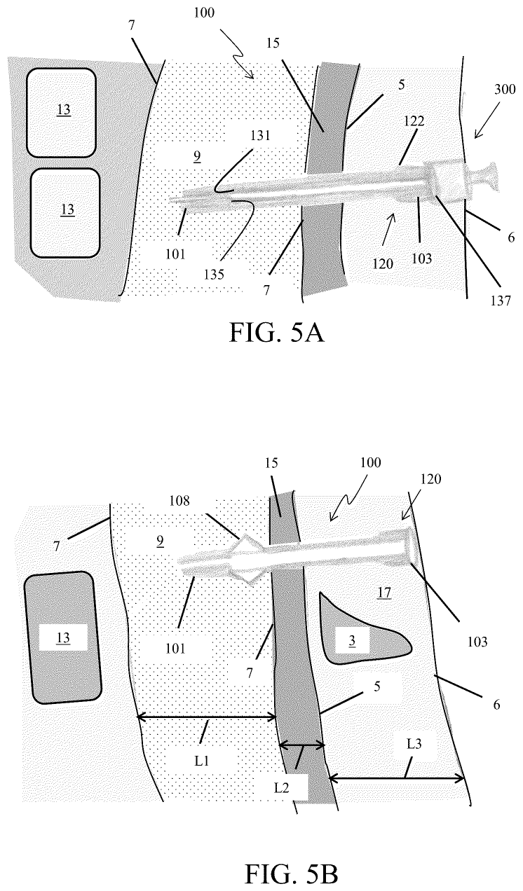

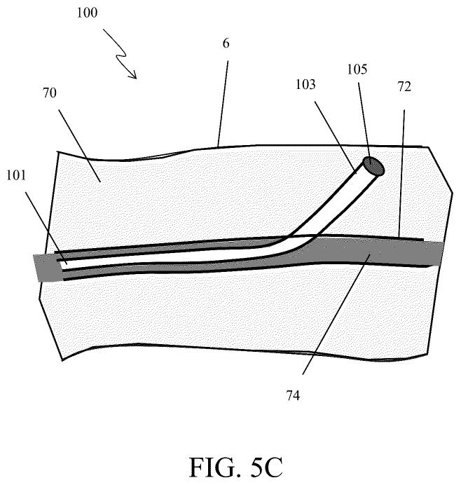

FIGS. 5A and 5B depict illustrative, non-limiting examples of a CSF-access device 100, according to some aspects of the present disclosure. In some arrangements, it may be desirable to arrange the CSF-access device 100 to have a port-like configuration that allows repeated access to the CSF space 9, as described herein. While the device 100 of the present disclosure is referred to, and described in the context of, a CSF-access device 100, the skilled artisan will appreciate the device 100 disclosed herein can be configured to access a blood vessel (e.g., artery, vein) instead of the CSF space. Thus, a CSF-access device 100 of the present disclosure encompasses a CSF-access device 100 configured to serve as a vascular port (e.g., venous port, arterial port) instead of CSF port.

FIG. 5A illustrates a port-configured CSF-access device 100 mounted onto a delivery assembly 300, according to some aspects of the present disclosure. In some arrangements, the delivery assembly 300 can be configured to implant the CSF-access device 100 such that a distal end 101 of the CSF-access device 100 resides within the CSF space 9 of the patient. In FIG. 5A, the delivery assembly 300 has been employed to advance the distal end 101 of the CSF-access device 100 toward the spine 13 through skin 6, through the ligamentum flavum 5, across the epidural space 15, through the dura 7, and into the CSF space 9 of the patient. As illustrated in FIG. 5B, after the port-configured CSF-access device 100 is properly implanted within the CSF space 9, the delivery assembly 300 can be removed from the CSF-access device 100, leaving the distal end 101 of the port-configured CSF-access device 100 within the CSF space 9 of the patient.

As shown in FIG. 5A, in some arrangements the port-configured CSF-access device 100 need not include a tissue anchor 108 (FIG. 5B). In some arrangements, the tissue anchor 108 can be configured to deploy upon removal of the delivery assembly 300 from the port-configured CSF-access device 100 after the CSF-access device 100 has been implanted. For example, as can be appreciated from FIGS. 5A and 5B, in some arrangements, the delivery assembly 300 can be configured to hold the CSF-access device 100 in tension when the CSF-access device 100 is mounted on the delivery assembly 300 (e.g., by a delivery device stop surface 131 pressing against an implant stop surface 135 and supported by a collar lock 137 at the proximal end 103). Upon release of the CSF-access device 100 from the delivery assembly 300, the CSF-access device 100 can be configured to retract the distal end 101 and the proximal end 103 toward one another (e.g., clastic recoil), and thereby buckling a distal portion of the lateral wall of the CSF-access device 100 to form a tissue anchor 108 (FIG. 5B). In some arrangements, however, the CSF-access device 100 can be configured to not have a tissue anchor 108 and to further not form a tissue anchor 108 upon release of the CSF-access device 100 from the delivery assembly 300.

In some arrangements, the CSF-access device 100 can include one or more tissue retention features configured to reduce migration of the CSF-access device 100, as described herein. For example, as indicated in FIG. 5A, the port-configured CSF-access device 100 can include near its proximal end 103 a proximal hub 120 that has a blunt, distal-facing flange 122, which can reduce or resist the CSF-access device 100 from migrating further toward the spine 13 following an implantation procedure. In some aspects, the CSF-access device 100 can include a flange 120 the reduces tissue migration of the CSF-access device 100 following implantation.

FIG. 5B illustrates that the port-configured CSF-access device 100 can be sized such that after implantation in a patient, a distal portion of the device 100 resides within a CSF space 9, having a transverse length indicated by L1 (e.g., a length between about 16 mm and about 18 mm), while an intermediate portion of the device 100 can reside within an epidural space 15, having a transverse length indicated by L2 (e.g., a length between about 5 mm and about 6 mm), while a proximal portion of the device can reside within a subcutaneous space 17, having a transverse length indicated by L3 (e.g., a length between about 3 centimeters and 5 centimeters, or greater than 5 centimeters for an obese patient).

FIG. 5C illustrates that the port-configured access device 100 sized for implantation into a intravascular space of a patient, according to some aspects of the present disclosure. In FIG. 5C, the access device 100 is shown with the distal end 101 residing within a luminal space 74 of a vein 72 in an arm 70 of the patient. The proximal end 103 of the access device 103 can be sutured in place under the skin 6 and include a sealing membrane 105, as described herein.

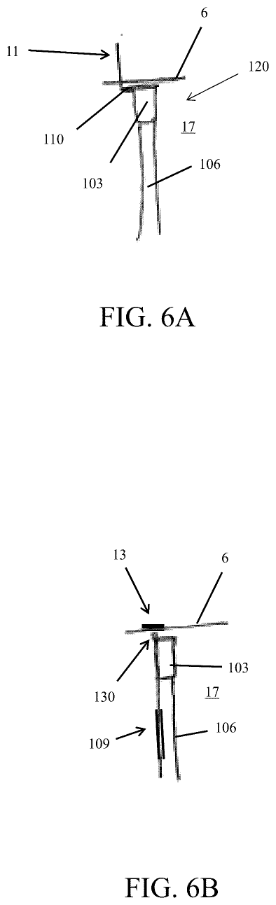

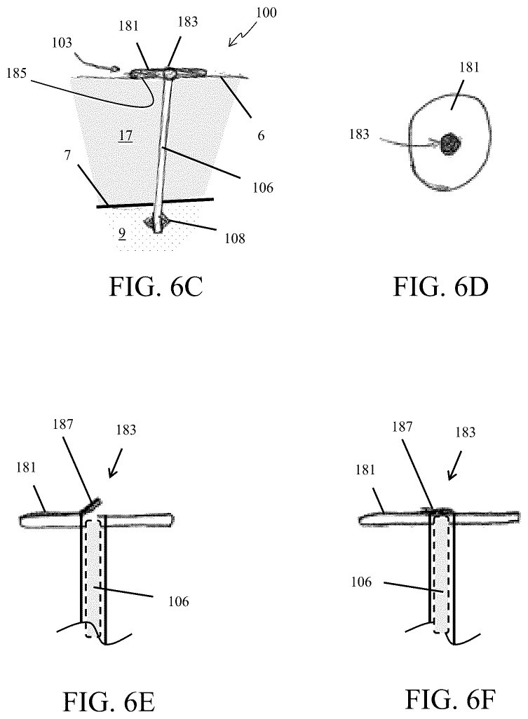

FIGS. 6A and 6B depict illustrative, non-limiting examples of a CSF-access device 100, according to some aspects of the present disclosure. As illustrated in FIG. 6A, the CSF-access device 100 can include a distal end 103 configured for subcutaneous implantation just below the outer layer of the patient's skin 6. A suture 11 can be passed through a suture ring 110 to hold the distal end 103 in place with the suture 11 marking the location of the distal end 103, thereby facilitating the repeatable locating and accessing of the CSF-access device 100, for example to periodically sample or drain the CSF of the patient in whom the device 100 is implanted.

FIG. 6B illustrates that the CSF-access device 100 can include a locating marker 130. The locating marker 130 can be echogenic, radioopaque, magnetic, or otherwise configured to assist in visualizing the locating marker 130 by a medical imaging technique. The locating marker 130 can facilitate locating or guiding the proximal end 103 with ultrasound, fluoroscopy, or other suitable medical imaging, or with a magnetic device. In some aspects, a marking tattoo 13 can be applied to a portion of the skin 6 that covers the proximal end 103. The marking tattoo 13 can assist in locating the proximal end 103. FIG. 6B further illustrates that the CSF-access device 100 can include a tissue-integration-promoting region 109, which can allow the proximal end 103 to be restricted from migration without requiring a suture 11 or in addition to having a suture 11.

FIGS. 6C-6F depict illustrative, non-limiting examples of a proximal end 103 of a port-configured CSF-access device 100, according to some aspects of the present disclosure. FIG. 6C illustrates the proximal end 103 can include an outer flange 181 that circumferentially surrounds a central access cap 183. The access cap 183 can be configured to seal access to the central conduit 106, for example by including a re-scalable capping membrane, as described herein. The outer flange 181 can include an adhesive disposed on a skin-facing surface 185 of the flange 181. FIG. 6D depicts a top view (i.e., a plan view of the skin from outside of the patient) of the proximal end 103 of FIG. 6C. FIGS. 6E and 6F illustrate that, in some aspects, the proximal end 103 can include a cover 187 that can alternate between an open configuration (FIG. 6E) and a closed configuration (FIG. 6F). In the open configuration, the cap 187 can allow access to the central conduit 106. In the closed configuration, the cap 187 can block access to the central conduit 106.

### Drain Configuration

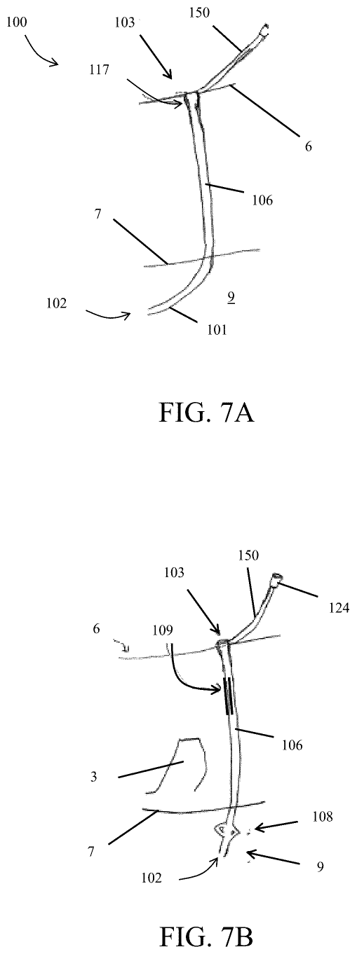

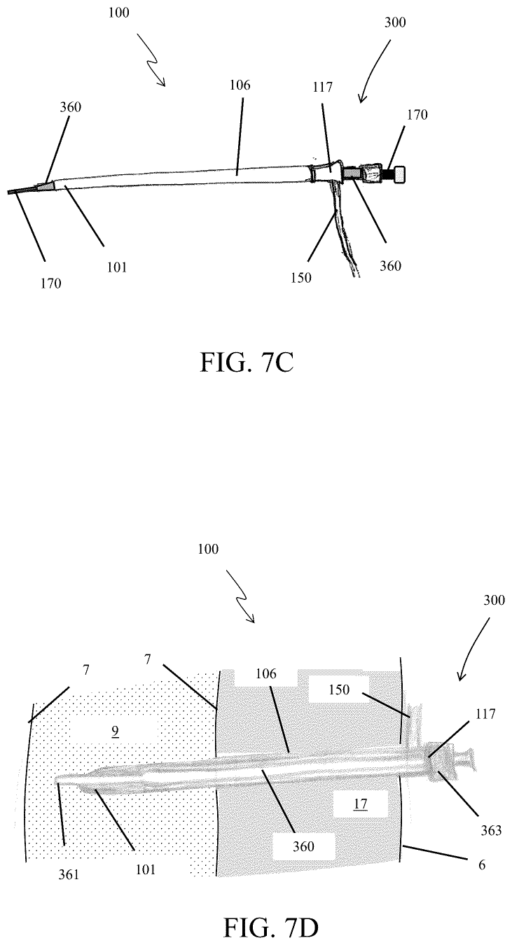

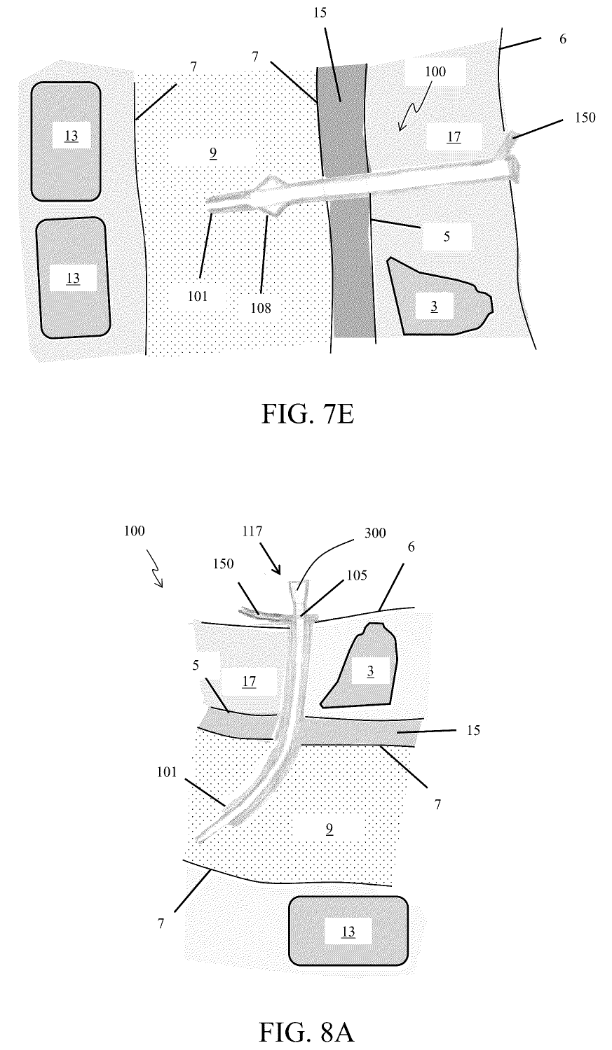

FIGS. 7A and 7B depict illustrative, non-limiting examples of a CSF-access device 100, according to some aspects of the present disclosure. In some arrangements, the CSF-access device 100 can be configured as a drain that allows CSF from the CSF space 9 to flow out of the CSF space 9. FIG. 7A shows the CSF-access device 100 can include a proximal hub 117 configured to fluidically connect an outflow conduit 150 to the central conduit 106 such that a flow path is established from the CSF space 9 to a space that is outside of the skin 6 of the patient. The proximal hub 117 can be configured to anchor (e.g., by suture, by adhesive) the device 100 to an outside surface of the skin 6.

As indicated in FIG. 7B, the outflow conduit 150 can terminate at proximal-end connector 124. The proximal-end connector 124 can include a fitting (e.g., a multi-position stopcock) that allows repeated access to the central conduit 106 and the CSF space 9 without requiring repeated puncturing of the dura 7. In some arrangements, the proximal-end connector 124 can include a scaling membrane 105 (FIG. 3) to enable repeated access to the central conduit 106 and CSF space 9 without requiring a repeated puncturing of the dura 7. FIG. 7B further illustrates that the CSF-access device 100 can be configured to include both a distal tissue anchor 108 and a proximal hub 117 that is expressed external to the patient's skin 6. Additionally and alternatively, the CSF-access device 100 can have a tissue-integration-promotion region 109 configured to promote the patient's tissue attachment to the device and thereby reduce migration of the device 100 following implantation.

FIG. 7C illustrates a CSF-access device 100 configured as a drain and mounted onto a delivery assembly 300, according to some aspects of the present disclosure. In some arrangements, the delivery assembly 300 can be configured to implant the CSF-access device 100 such that a distal end 101 of the CSF-access device 100 resides within the CSF space 9 of the patient, as described herein. The delivery assembly 300 can be removed from the CSF-access device 100, leaving the distal end 101 of the CSF-access device 100 in place within the CSF space 9 of the patient and allowing the CSF-access device 100 to serve as a drain that establishes access to the CSF space 9 without requiring repeated puncture of the dura 7. The delivery assembly 300 of FIG. 7C includes a co-axial arrangement of a delivery stylet 170 within a delivery needle 360, which in turn is within the central conduit 106 of the CSF-access device 100. The CSF-access device 100 can be mounted over the delivery assembly 300 and inserted together with the delivery assembly 300 as one unit like an insertion method for an intravenous catheter. In some arrangements, the proximal hub 117 can be rotationally moveable relative to the central conduit 106, allowing the proximal hub 117 to spin and accommodate a rotational movement of the outflow conduit 150. This can allow the outflow conduit 150 to be positioned as desired without altering a position of the distal end 101 of the CSF-access device 100.

FIG. 7D is a cross-sectional view of a CSF-access device 100 mounted on a delivery assembly 300, illustrating that in some arrangements, the delivery assembly 300 can comprise a delivery needle 360 alone without a delivery stylet 170. In some arrangements, the CSF-access device 100 can be implanted via a Seldinger technique in which a spinal needle (not shown) surrounds an inner coaxial stylet (not shown) to access the CSF space 9. The inner stylet can then removed from the bore of the spinal needle and replaced with a wire that is inserted through a proximal end of the spinal needle. In some arrangements, the wire can have a soft tip that grows gradually stiffer along a longitudinal length of the wire in the proximal direction. Once the wire is in place in the CSF space 9, the spinal needle can be removed and exchanged for a CSF-access device 100, which can be introduced over-the-wire mounted on a delivery assembly 300 (e.g., delivery needle 360). Once the CSF-access device 100 is properly placed with access to the CSF-space 9, the delivery assembly 300 and wire (not shown) can then be removed from the CSF-access device 100. In some arrangements, the delivery needle 360 can include a distal tip 361 that is configured to pierce the dura 7. In some aspects, the delivery needle 360 can have a proximal end 363 configured to reversibly interlock with the proximal hub 117, such as, for example by a quarter-turn thread or a luer-lock like coupling. After implanting the CSF-access device 100 with the delivery needle 360, the delivery needle 360 can be unlocked from the CSF-access device 100 (e.g., by turning the delivery needle 360 a quarter turn to unlock the proximal end 363 from the proximal hub 117), allowing the delivery needle 360 to be removed while leaving the CSF-access device 100 implanted undisturbed in the CSF space 9.

As illustrated in FIG. 7E, after the drain-configured CSF-access device 100 is properly implanted within the CSF space 9, the delivery assembly 300 can be removed from the CSF-access device 100, leaving the distal end 101 of the CSF-access device 100 in place within the CSF space 9 of the patient. In some arrangements, removing the delivery assembly 300 from the CSF-access device 100 can deploy a tissue anchor 108, as described herein. After removal of the delivery assembly 300, the CSF-access device can establish a flow path through the central conduit 106 between the outflow conduit 150 and the distal tip 101 within the CSF space 9.

### Sheath Configuration

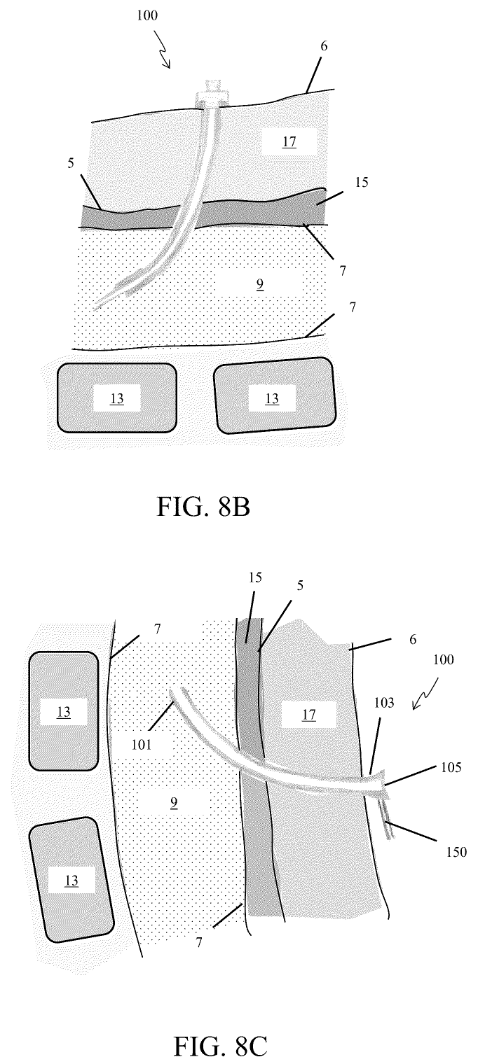

FIG. 8A depicts an illustrative, non-limiting example of the CSF-access device 100, according to some aspects of the present disclosure. In some arrangements, the CSF-access device can have a sheath-like configuration, providing a pathway to access the CSF space 9 for performing tasks such as, for example, imaging, biopsy, interventions, or deploying (e.g, inserting, retrieving) miniature robotic medical devices or electrodes (e.g., for a brain-computer interface). In some aspects, the sheath configuration of the CSF-access device 100 can provide a pathway from which to deploy an endoscope, for example for imaging purposes. In some arrangements, the sheath-configured CSF-access device 100 can provide a pathway from which to deploy an optical coherence tomography (OCT) device, an ultrasound probe, a fiber optic wire, or a camera. The sheath-configured CSF-access device 100 can further provide a pathway from which to deploy a biopsy device to perform a biopsy method such as, for example, a fine-needle aspiration, a punch biopsy, a core biopsy. In some aspects, the sheath-configured CSF-access device 100 can provide a pathway to perform interventions for addressing medical issues such as, for example, tumors, vascular malformations, stroke, neurodegenerative diseases, immunoinflammatory diseases, paralysis, CSF abnormalities (e.g., arachnoid cyst, hydrocephalus, adhesions).

FIG. 8A illustrates a sheath-configured CSF-access device 100 mounted on a delivery assembly 300 with the distal tip 101 of the device 100 positioned within a CSF-space 9 of the patient while the proximal hub 117 is disposed exteriorly of the patient′ skin 6. As shown in FIG. 8A, the proximal hub 117 can include a sealing membrane 105 through which passes the delivery assembly 300. The sealing membrane 105 can be configured to establish a fluidic scal over the central bore of the CSF-access device 100 upon removal of the delivery assembly 300 from the CSF-access device 100. The delivery assembly 300 can be made of a rigid material (e.g., metal or polymer) and shaped (e.g., beveled) to puncture intervening tissues as the distal end of the delivery assembly 300 is advanced into the CSF space 9, as described herein. The distal end 101 of the CSF-access device 100 can be angled and can be torqueable, enabling positioning of the distal end 101 within the CSF space 9. The proximal hub 117 can be configured to connect with an outflow conduit 150, as shown in FIG. 8A. FIG. 8B shows that in some arrangements, the proximal hub 117 can be configured to connect with no outflow conduit 150. FIG. 8C illustrates that, in some arrangements, the sheath-configured CSF-access device 100 can include a sealing membrane 105 and an outflow conduit 150. In FIG. 8C, the sheath-configured CSF-access device 100 is shown after the delivery assembly 300 (FIG. 8B) has been removed from the CSF-access device 100, leaving the CSF-access device 100 implanted in the patient with the distal end 101 in the CSF space 9 and the proximal end 103 disposed at the patient's skin 6.

### Shunt Configuration

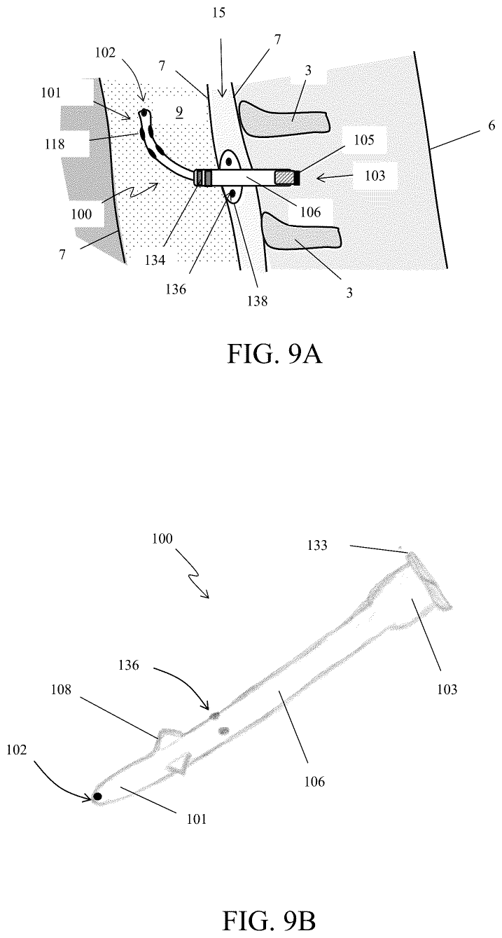

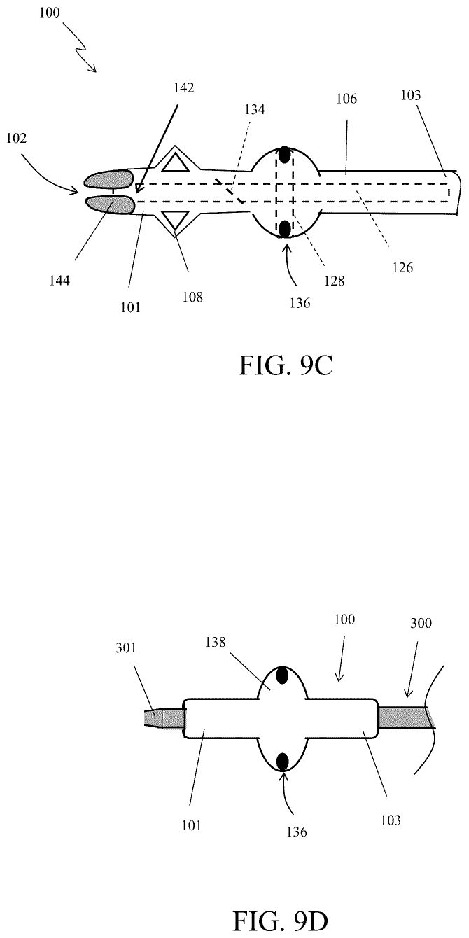

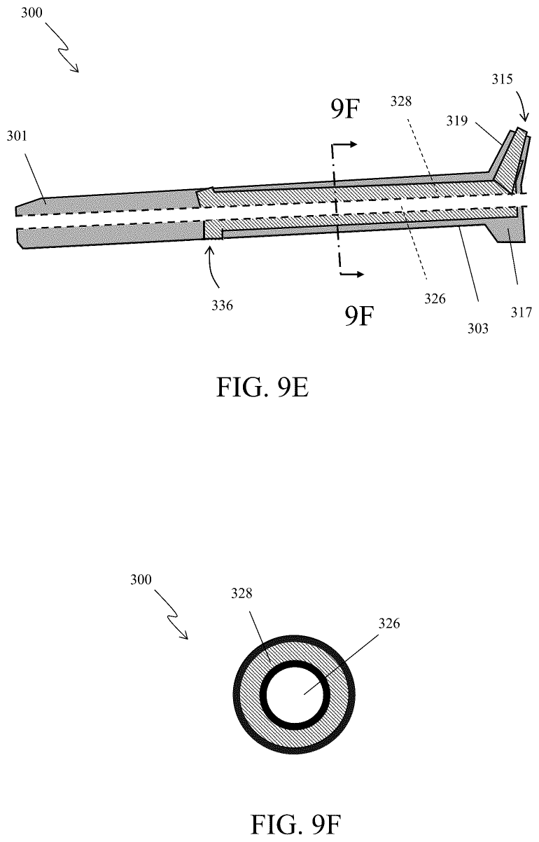

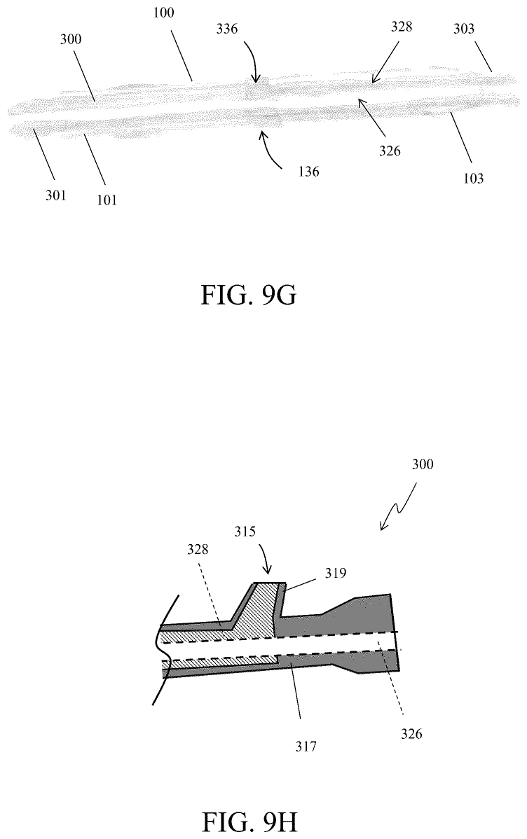

FIG. 9A depicts an illustrative, non-limiting example of a shunt-configured arrangement of the CSF-access device 100, according to some aspects of the present disclosure. The CSF-access device 100 of FIG. 9A is shown implanted in a patient to access a CSF space 9 of the patient. The CSF-access device 100 can be sized so that a distal end 101 resides in the CSF space 9 while a shunting port 136 resides within the epidural space 15. In some arrangements, the CSF-access device 100 can shunt CSF from the CSF space 9 to the epidural space 15 through the shunting port 136, as described herein. In some aspects, the CSF-access device 100 can be sized to shunt CSF fluid from the CSF space 9 to a location other than the epidural space 15 (e.g. to a location within a muscle layer or a subcutaneous space).

As shown in FIG. 9A, the shunting port 136 can be disposed on a blocking structure 138 that protrudes radially outward from the central axis of the central conduit 106. The blocking structure 138 can increase the diameter of the CSF-access device 100 and thereby block the device from advancing the shunting port 136 past the dura 7 and into the CSF space 9. The CSF-access device 100 can include a one-way valve 134 that allows fluid to flow in a distal-to-proximal direction while inhibiting flow in a proximal-to-distal direction, as described herein. The CSF fluid can enter the center bore of the device 100 at the distal end 101 through either a terminal opening 102 or a lateral opening 118. CSF fluid can flow through the one-way valve 134 and exit the device 100 through the shunting port 136 to enter the epidural space 15, thereby establishing a shunting flow path for CSF through the CSF-access device 100. FIG. 9A further illustrates that the proximal end 103 can include a device-retrieval docking structure 105 configured to assist with retrieval and removal of the device 100 from the patient, as described herein.

FIG. 9B illustrates a CSF-access device 100 configured as a shunt, according to some aspects of the present disclosure. In some arrangements, the shunting port 136 can be disposed on a lateral surface of the device 100 at a radius similar to that of the central conduit 106. In other words, the shunting port 136 need not be required to be disposed on a blocking structure 138 (as shown in FIG. 9A) but can be disposed directly through the central conduit 106. FIG. 9B further illustrates that the CSF-access device 100 can include a delivery-docking feature 133 at the proximal end 103. The delivery-docking feature 133 can be configured as a coupling (e.g., luer lock) that assists with coupling the CSF-access device 100 to a delivery apparatus 300 for implantation of the CSF-access device 100 within a patient, as described herein.

FIG. 9C illustrates a CSF-access device 100 configured as a shunt, according to some aspects of the present disclosure. In some aspects, the CSF-access device 100 can include a central bore 126 that extends from the proximal end 103 to the distal end 101. As described herein, the central bore 126 can be used to mount the CSF-access device 100 onto a delivery apparatus 300 for implanting the device 100 into a patient. After implantation of the CSF-access device 100, the delivery apparatus 300 can be decoupled from the device 100 and withdrawn from the central bore 126, making the central bore 126 available for conveying fluid (e.g., CSF) through the CSF-access device 100. The CSF-access device 100 can include a lateral channel 128 that establishes a flow pathway between the shunting port 136 and the central bore 126. In some arrangements, the CSF-access device 100 can include a one-way valve 134 within the central bore 126 and configured to allow flow through the one-way valve 134 only in a distal-to-proximal direction. The distal tip 101 can include a marker band 144 that can be made of a material that presents a prominent profile for imaging purposes (e.g., an echogenic material). The marker band 144 can assist with positioning of the CSF-access device 100 within the patient during implantation of the device 100. In some aspects, the central bore 126 can include a restriction at the distal tip that forms a proximal-facing stop surface 142. As described herein, the proximal-facing stop surface 142 can be sized to limit the extent the delivery apparatus 300 may advance distally through the CSF-access device 100. In some aspects, the proximal-facing stop surface 142 can intrude only partially into the central bore 126, leaving a centermost passageway that can be sized to allow a guidewire (not shown) to extend distally through the distal-end opening 102. In some arrangements, the size of the device 100 and the central bore 126 can be selected to achieve various fluid flow rates (e.g., a high-flow rate device 100 may have a central bore 126 that has a larger diameter compared to that of a low-flow rate device 100).

FIG. 9D illustrates a CSF-access device 100 configured as a shunt and mounted onto a delivery assembly 300, according to some aspects of the present disclosure. In some aspects, the delivery assembly 300 can be configured to implant the CSF-access device 100 such that a distal end 101 of the CSF-access device 100 resides within the CSF space 9 of the patient. In some aspects, the delivery assembly 300 can be removed from the CSF-access device 100, leaving the distal end 101 of the CSF-access device 100 in place within the CSF space 9 of the patient and allowing the CSF-access device 100 to serve as a shunt. As illustrated in FIG. 9D, the delivery assembly 300 can include a distal tip 301 that has a blunt profile.

FIG. 9E depicts an illustrative, non-limiting example of a delivery assembly 300 for use in implanting a shunt-configured CSF-access device 100, according to some aspects of the present disclosure. As illustrated in FIG. 9E, after the delivery assembly 300 can include an axial bore 326 and a lateral passageway 328 disposed radially outward of the axial bore 326. The delivery assembly 300 can include lateral ports 336 that connect fluidically with the lateral channel 328. In some aspects, the lateral port 336 shape and number can be varied according to the desired flow dynamics for the device 100. For example, larger or more numerous lateral ports 336 can be used in some arrangements to achieve a high flow rate through the CSF-access device 100. In some arrangements, the delivery assembly 300 can be sized to align the lateral ports 336 with the shunting ports 136 of the CSF-access device 100 when the device 100 is mounted on the delivery assembly 300. FIG. 9F is an axial cross-sectional view of the delivery assembly 300 from the view indicated in FIG. 9E. As shown in FIG. 9F, the delivery assembly 300 can have a lateral channel 328 that is disposed coaxial with, and radially outward from, the axial bore 326. The delivery assembly 300 can further include a proximal hub 317 that includes a branch port 319 that defines a side-port passageway 315 that connects fluidically with the lateral channel 328, as shown in FIG. 9E.

FIG. 9G depicts an illustrative, non-limiting example of the shunt-configured arrangement of the CSF-access device 100, according to some aspects of the present disclosure. FIG. 9G depicts a cross-sectional view of the shunt-configured CSF-access device 100 mounted on a delivery assembly 300. In some aspects, the lateral ports 336 are in fluid communication with the shunting ports 136 when the CSF-access device 100 is mounted on the delivery assembly 300. In this way, contrast or air can be introduced through the side-port passageway 315 (FIG. 9E) to exit the lateral channel 328 at the lateral port 336 and pass through the shunting port 136 of the CSF-access device 100 and thereby assist in placement of the CSF-access device 100 within the patient. In some arrangements, the shunting port 136 can be distal or proximal to the lateral port 336 and in fluid communication with the lateral port 336. In other words, the shunting port 136 need not align longitudinally with the lateral port 336 when the device 100 is mounted on the delivery assembly 300.

FIG. 9H depicts an illustrative, non-limiting example of a proximal hub 317 of a delivery assembly 300, according to some aspects of the present disclosure. As shown in FIG. 9H, the proximal hub 317 can include a branch port 319 that provides access to the lateral channel 328 through the side-port passageway 315.

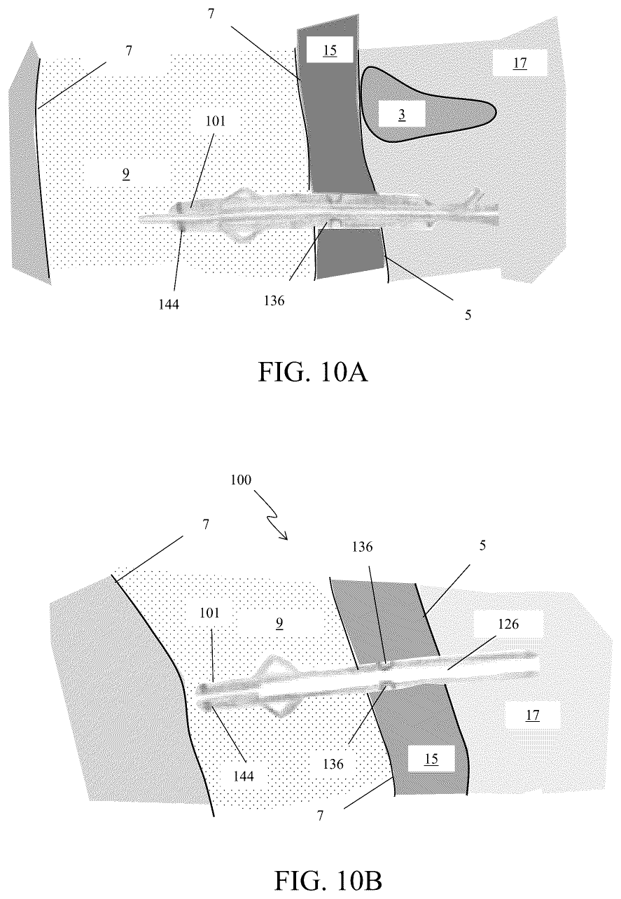

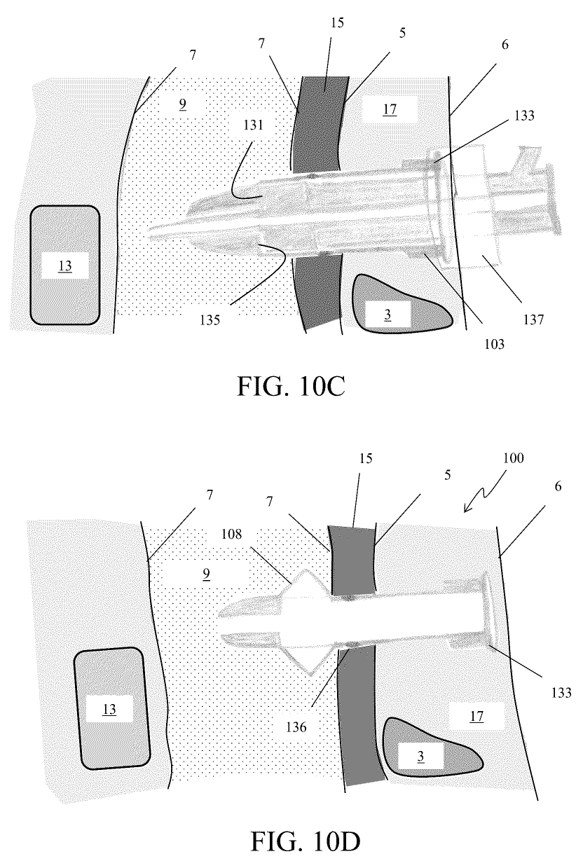

FIGS. 10A and 10B depict an illustrative, non-limiting example of a shunt-configured CSF-access device 100, according to some aspects of the present disclosure. In FIG. 10A, the CSF-access device 100 is shown implanted in a patient with the delivery assembly 300 still coupled to the CSF-access device 100. FIG. 10B illustrates the CSF-access device 100 of FIG. 10A after the delivery assembly 300 has been decoupled from and removed from the CSF-access device 100. The CSF-access device 100 can be sized so that the shunting ports 136 reside in the epidural space 15 while the distal tip 101 resides in the CSF space 9. Imaging-brilliant marker bands can be disposed at the distal tip 101 to assist with placement of the CSF-access device 100. As can be appreciated from FIG. 10B, after completing an implantation method of the device 100, the CSF-access device 100 can provide a shunting flow pathway from the CSF space 9, along the central bore 126 of the device 100 in a distal-to-proximal direction, exiting the device 100 through the shunting ports 136 to enter the epidural space 15.

FIGS. 10C and 10D depict an illustrative, non-limiting example of a shunt-configured CSF-access device 100, according to some aspects of the present disclosure. In FIG. 10C, the CSF-access device 100 is shown implanted in a patient with the delivery assembly 300 still coupled to the CSF-access device 100. FIG. 10D illustrates the CSF-access device 100 of FIG. 10C after the delivery assembly 300 has been decoupled from and removed from the CSF-access device 100. As can be appreciated from FIGS. 10C and 10D, the shunt-configured CSF-access device 100 can include a tissue anchor 108 that deploys as the delivery docking feature 133 of the device 100 is disengaged from the collar lock 137 of the delivery assembly 300, as described herein.

### Pump Configuration

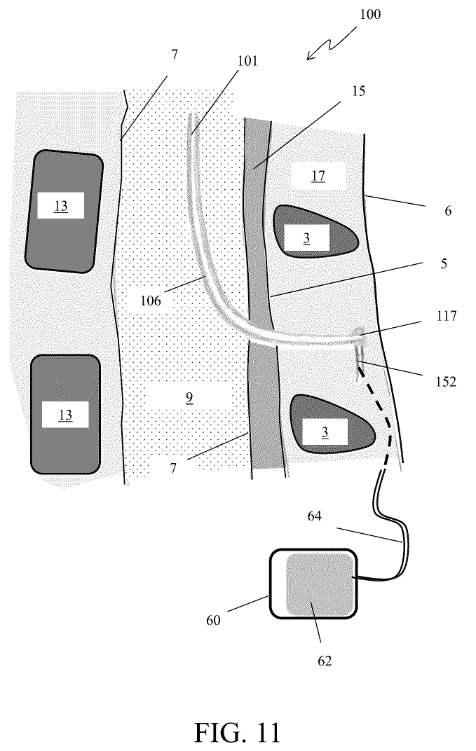

FIG. 11 depicts an illustrative, non-limiting example of a CSF-access device 100 configured for use as a pump for injecting fluid into a CSF space 9, according to some aspects of the present disclosure. The pump-configured CSF-access device 100 can include a pump-side conduit 152. The downstream end of the pump-side conduit 152 can connect to the proximal hub 117 and form a fluidic connection with the central conduit 106 of the CSF-access device 100. The upstream end of the pump-side conduit 152 can connect to an intrathecal pump 60 through an upstream conduit 64. The intrathecal pump 60 can pump fluid 62 (e.g., a flowable therapeutic injectate) into the upstream conduit 64 and on into the pump-side conduit 152. The intrathecal pump 60 can further propel the pumped fluid 62 along the central conduit 106, through the distal end opening 101, and into the CSF space 9.

### Device Retrieval

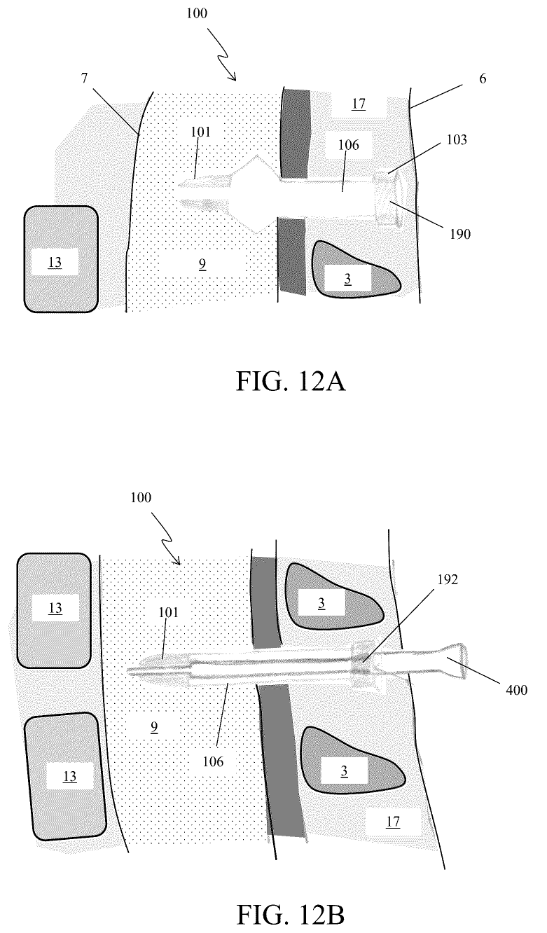

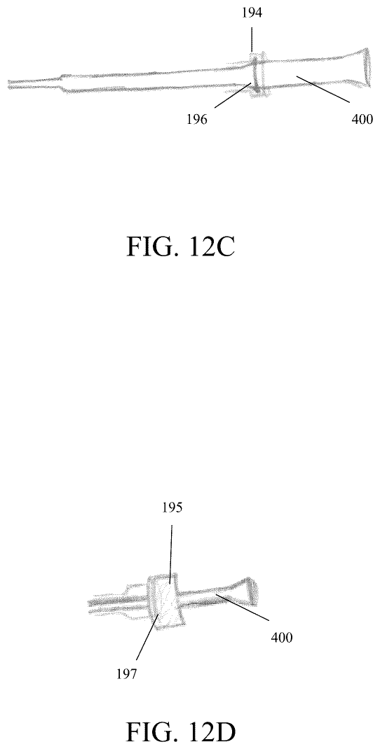

FIGS. 12A and 12B depict an illustrative, non-limiting example of a CSF-access device 100 configured for facilitating retrieval of the CSF-access device 100 from the patient, according to some aspects of the present disclosure. As shown in FIG. 12A, the proximal end 103 of the CSF-access device 100 can include a retrieval feature at the proximal end 103 of the device 100. The retrieval feature depicted in FIG. 12A is a magnetic collar 190 that is configured to align with and magnetically attract a corresponding magnetic ring 192 (FIG. 2B) disposed on a retrieval device 400 when the retrieval device 400 is inserted into the central conduit 106 of the CSF-access device 100. FIG. 12C illustrates that the retrieval feature of the CSF-access device 100 can be configured as one or more keyed grooves 194 disposed on the device 100 that connect to a corresponding one or more ridges 196 disposed on the retrieval device 400. FIG. 12D illustrates that in some arrangements, the retrieval feature of the CSF-access device 100 can be configured as a luer lock 195 disposed on the retrieval device 400 that connect to a corresponding collar 197 disposed on the CSF-access device 100.

### Closure Device

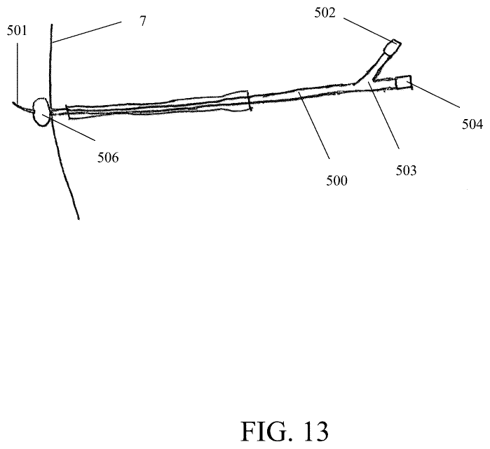

FIG. 13 depicts an illustrative, non-limiting example of a closure device 500 for sealing a dura 7 of a patient following removal of the CSF-access device 100 from the patient, according to some aspects of the present disclosure. The closure device 500 can include a balloon port 502 and an injection port 504. The balloon port 502 can be fluidically connected to a balloon 506 at a distal end 501 of the closure device 500. Inflation and deflation of the balloon 506 can be controlled by introducing and withdrawing fluid (e.g., saline, air) at the proximal end 503 of the closure device 500 through the balloon port 502. Tissue sealant can be expelled from an outflow orifice disposed on an outer surface of the distal end of the closure device, the distal orifice being fluidically connected to the injection port 504. In some aspects, the inflation of the balloon 506 can allow for confirmation that the CSF-access device 100 is in the CSF space. The balloon 506 can be inflated with various materials (e.g., saline, contrast). The balloon 506 can ensure closure of the dural hole during injection of a sealant material in the epidural space. The balloon 506 can be deflated and removed, for example, after the sealant material has been given opportunity to cure.

### Device Delivery

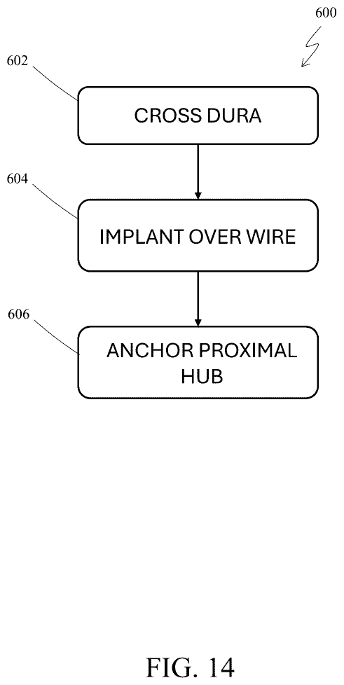

FIG. 14 depicts an illustrative, non-limiting method for implanting a CSF-access device 100 into a patient 2 to gain access to the CSF space 9, as described herein. The method 600 can include a wire placement step in which a distal end of a delivery wire is delivered to across the dura 7 of the patient and into the CSF space 9. The method 600 can further include a implant placement step in which with a distal end of the CSF-access device 100 is advanced along the placed wire, thereby bringing the distal end 101 of the CSF-access device 100 into the CSF space 9 of the patient. The method 600 can further include a securement step 606 in which the delivery assembly 300 is removed from the patient 2, leaving the distal end 101 in place within the CSF space), and securing the proximal end 103 to a skin 6 of the patient 2 to restrict movement of the proximal end 103, as described herein.

### Other Variations and Terminology

While certain embodiments have been described, these embodiments have been presented by way of example only and are not intended to limit the scope of protection. Indeed, the novel methods and systems described herein may be embodied in a variety of other forms. It will be understood by those skilled in the art that the present disclosure extends beyond the specifically disclosed embodiments to other alternative embodiments or uses and obvious modifications and equivalents thereof, including embodiments which do not provide all of the features and advantages set forth herein. Furthermore, various omissions, substitutions, and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed; others may be added. Accordingly, the scope of the present disclosure is not intended to be limited by the specific disclosures of preferred embodiments herein and may be defined by claims as presented herein or as presented in the future. The language of the claims is to be interpreted broadly based on the language employed in the claims and not limited to the examples described in the patent specification of during prosecution of the application, which examples are to be construed as non-exclusive.

Features, materials, characteristics, or groups described in conjunction with a particular aspect, embodiment, or example are to be understood to be applicable to any other aspect, embodiment, or example described herein unless incompatible therewith. All of the features disclosed in this specification (including any accompanying claims, abstract, and drawings), or all of the steps of any method or process so disclosed, may be combined in any combination, except combinations where at least some of such features or steps are mutually exclusive. The protection extends to any novel one, or any novel combination, of the features disclosed in this specification (including any accompanying claims, abstract, and drawings), or to any novel one, or any novel combination, of the steps of any method or process so disclosed.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of the stated amount. As another example, the terms “generally parallel” and “substantially parallel” may refer to a value, amount, or characteristic that departs from exactly parallel by less than 15 degrees.

What is claimed is:

1. An apparatus comprising a CSF-access device mounted on a delivery assembly, the apparatus further comprising:
a central bore extending from a proximal end of the device to a distal end of the device;
a shunting port disposed in a wall of the device and connected fluidically to the central bore by a lateral passageway disposed in the wall of the device;
an axial bore extending from a proximal end of the delivery assembly to a distal end of the delivery assembly; and
a lateral channel disposed within the delivery assembly and radially outward of the axial bore, wherein the axial bore is isolated fluidically from the lateral channel.

2. The apparatus of claim 1, further comprising an epidural-blocking structure disposed on the device, the shunting port disposed on the epidural-blocking structure.

3. The apparatus of claim 1, further comprising a tissue anchor disposed on the distal end of the device.

4. The apparatus of claim 3, wherein the epidural-blocking structure and the tissue anchor are separated from one another by a longitudinal length along the device, the longitudinal length being within the range of 1 mm and 6 mm.

5. The apparatus of claim 3, further comprising a collar lock disposed on the delivery assembly and configured to hold the proximal end of the device and further configured to prevent a stop service on the device from advancing proximally past a corresponding stop service disposed on the delivery assembly.

6. The apparatus of claim 5, wherein a radial profile of the tissue anchor increases upon the collar lock and the proximal end of the device being decoupled from one another.

7. The apparatus of claim 6, further comprising a one-way valve disposed in the central bore and configured to allow flow along the central bore in a direction from the distal end to the proximal end.

8. A device for accessing a cerebrospinal fluid of a patient, the device comprising:
a central conduit that defines a central bore, the central bore extending from a proximal end of the device to a distal end of the device;
a shunting port disposed on an outer surface of the central conduit and fluidically connected to the central bore; and
a sealing membrane disposed at the proximal end, the sealing membrane configured to form a fluidic seal of the central bore at the proximal end of the central conduit.

9. The device of claim 8, further comprising a one-way valve disposed within the central bore, the one-way valve configured to block a flow of a fluid in the central bore in a direction from the proximal end to the distal end.

10. The device of claim 8, further comprising a tissue anchor disposed on the central conduit, the tissue anchor being disposed longitudinally between the shunting port and the distal end.

11. The device of claim 8, further comprising a device stop surface disposed within the central bore, the device stop surface being disposed longitudinally between the shunting port and the distal end.

12. The device of claim 8, further comprising an echogenic marker disposed at a proximal hub of the device, the proximal hub comprising the sealing membrane.

13. The device of claim 8, further comprising a suture ring disposed at a proximal hub of the device, the proximal hub comprising the sealing membrane.

14. The device of claim 8, further comprising an outflow conduit disposed at a proximal hub of the device, the outflow conduit in fluidic communication with the central bore.

15. The device of claim 8, further comprising a magnetic collar disposed at a proximal hub of the device, the proximal hub comprising the sealing membrane.

16. A device for accessing a cerebrospinal fluid of a patient, the device comprising:
a central bore defined by a central conduit, the central bore extending from a distal end opening of the device to a proximal hub of the device;
an outflow conduit disposed at the proximal hub, the outflow conduit connected fluidically with the central bore;
a pump fluidically connected to the outflow conduit by an upstream conduit, the pump configured to pump a pumping fluid through the upstream conduit and into the outflow conduit.

17. The device of claim 16, further comprising a stop surface extending from the central conduit into the central bore.

18. The device of claim 16, further comprising a sealing membrane disposed over a proximal end opening of the device.

19. The device of claim 18, further comprising an echogenic marker disposed at the proximal end of the device.

20. The device of claim 19, further comprising magnetic collar disposed at the proximal end of the device.
